(19) United States
(12) Patent Application Publication
(10) Pub. No.: US 2025/0205181 A1
(43) Pub. Date: Jun. 26, 2025

(54) ANANDAMIDE CYCLODEXTRIN INCLUSION COMPLEX VEHICLES

(71) Applicant: CZAP RESEARCH AND DEVELOPMENT, LLC, Napa, CA (US)

(72) Inventor: Al Czap, Napa, CA (US)

(21) Appl. No.: 18/294,821

(22) PCT Filed: Jul. 15, 2022
(86) PCT No.: PCT/IB2022/056550

§ 371 Date: Feb. 2, 2024

Related U.S. Application Data
(60) Provisional application No. 63/229,447, filed on Aug. 4, 2021.

(51) Int. Cl. A61K 31/164; A61K 9/48; A61K 31/198; A61K 38/47; A61K 47/69; A61P 15/10

(52) U.S. Cl. CPC A61K 31/164; A61K 9/4808; A61K 9/4866; A61K 31/198; A61K 38/47; A61K 47/6951; A61P 15/10; C12Y 302/01001

(57) ABSTRACT

The invention provides oral cyclodextrin inclusion complex formulations in which an anandamide cyclodextrin inclusion complex is provided together with enzyme having a cyclodextrin-degrading activity capable of digesting the cyclodextrin, so that upon delivery of the formulation to a target tissue the enzyme is activated and releases the anandamide from the cyclodextrin cavity. In alternative aspects, these cyclodextrin inclusion complex formulations are provided in the form of time release formulations, treating or preventing a nitric oxide deficiency or a disease which can be treated or prevented by increasing endogenous nitric oxide levels in a mammal.

### FIELD OF THE INVENTION

The invention is in the field of biochemical constructs for delivery of lipidic cannabinoid receptor agonists as inclusions within cyclodextrins, in formulations that include enzymes having cyclodextrin-degrading activities.

### BACKGROUND OF THE INVENTION

Cyclodextrins are non-reducing cyclic glucose oligosaccharides, frequently the product of cyclomaltodextrin glucanotransferase (E.C. 2.4.1.19; CGTase) catalyzed degradation of starch. Cyclodextrins may have a variety of structures (see Saenger et al., Chem. Rev. 98 (1998) 1787-1802), including three common cyclodextrins with 6, 7 or 8 D-glucopyranonsyl residues (α-, β-, and γ-cyclodextrin respectively) linked in a ring by α-1,4 glycosidic bonds. The frustoconical shape of cyclodextrins forms a cavity or lumen, with the cavities having different diameters depending on the number of glucose units. The scale of selected cyclodextrin (CD) structures is set out in Table 1. Larger cyclodextrins such as cyclomaltononaose (δ-CD) and cyclomaltodecaose (ϵ-CD) are also possible, as well as a variety of cyclodextrin-based supra-molecular structures (see Zhang and Ma, Adv Drug Deliv Rev. 2013 August; 65 (9): 1215-33).

TABLE 1
cyclodextrin structures

Lumen diameter (nm)

| Cyclodextrin | Inner rim | Outer rim |
|---|---|---|
| α, (glucose)₆ | 0.45 | 0.53 |
| β, (glucose)₇ | 0.60 | 0.65 |
| γ, (glucose)₈ | 0.75 | 0.85 |

Cyclodextrins are generally amphipathic, with the wider rim of the lumen displaying the 2- and 3-OH groups and the narrower rim displaying 6-OH. These hydrophilic hydroxyl groups are accordingly on the outside of the lumen, whereas the inner surface is generally hydrophobic and lined with the anomeric oxygen atoms and the C3-H and C5-H hydrogen atoms. In aqueous solution, this hydrophobic lumen may contain water molecules, for example about 3 (α-CD), 7 (β-CD) or 9 (γ-CD) poorly held but low entropy, and hence relatively easily displaceable water molecules. Thus, otherwise hydrophilic cyclodextrins may bind retain one or more suitably-sized molecules within, or partially within, the lumen of the CD, forming a cyclodextrin inclusion body or complex. For example, non-polar aliphatic and aromatic compounds, including drugs, such as lipophilic drugs, may be bound so as to increase the water solubility of normally hydrophobic compounds or minimize undesirable properties such as odor or taste in certain food additives. For this reason, cyclodextrin inclusions are widely used in the pharmaceutical, food and cosmetic fields (see Hedges, Chem. Rev. 98 (1998) 2035-2044). Cyclodextrins have for example been used in a variety of sustained release drug preparations, such as for inclusion complexes of a medical compound with a hydrophobic cyclodextrin derivative (U.S. Pat. No. 4,869,904).

Cyclodextrins may be chemically modified in a wide variety of ways. For example, to modify the inclusion specificity, physical and chemical properties of the cyclodextrin. Hydroxyl groups of a CD may for example be derivatized. For example, two modified CDs have been used in a number of pharmaceutical products: SBE-β-CD, or Captisol, a polyanionic variably substituted sulfobutyl ether of β-CD, and HP-β-CD, a modified CD commercially developed by Janssen. Additional CD derivatives include sugammadex or Org-25969, in which the 6-hydroxy groups on γ-CD have been replaced by carboxythio acetate ether linkages, and hydroxybutenyl-β-CD. Alternative forms of cyclodextrin include: 2,6-Di-O-methyl-β-CD (DIMEB), 2-hydroxylpropyl-β-cyclodextrin (HP-β-CD), randomly methylated-β-cyclodextrin (RAMEB), sulfobutyl ether β-cyclodextrin (SBE-β-CD), and sulfobutylether-γ-cyclodextrin (SBEγCD), sulfobutylated beta-cyclodextrin sodium salt, sulfobutylated beta-cyclodextrin sodium salt, (2-Hydroxypropyl)-alpha-cyclodextrin, (2-Hydroxypropyl)-beta-cyclodextrin, (2-Hydroxypropyl)-gamma-cyclodextrin, DIMEB-50 Heptakis(2,6-di-O-methyl)-beta-cyclodextrin, TRIMEB Heptakis(2,3,6-tri-O-methyl)-beta-cyclodextrin, methyl-beta-cyclodextrin, octakis(6-deoxy-6-iodo)-gamma-cyclodexrin, and, octakis(6-deoxy-6-bromo)-gamma-cyclodexrin. Although CDs such as these have been developed with favorable pharmacological and toxicological profiles, there is the potential that, following administration, residual CDs may perturb the pharmacokinetic properties of drugs, including coadministered drugs, particularly after parenteral administration (see Stella and He, Toxicol Pathol January 2008 vol. 36 no. 1 30-42).

Cyclodextrins are variably susceptible to enzymatic digestion. For example, γ-CD is relatively easily hydrolyzed by α-amylases whereas α-cyclodextrin is more poorly hydrolyzed. CD based therapeutics generally depend on the activity of endogenous amylases to digest the CD. There is however significant variability in amylase activity between patients. For example, patients with pancreatic insufficiency, cystic fibrosis, celiac disease or Crohn's disease, may lack normal amounts of amylase. Similarly, patients, particularly geriatric patients, may be deficient in gastric acid production and thereby fail to create conditions of appropriately low pH in the duodenum to properly trigger release of pancreatic amylase. A similar effect may result from the increasing common use of antacids, histamine-2 blockers, proton pump inhibitors or alternative acid blockers.

A variety of microbial cyclodextrin digesting enzymes have been identified. CD-degrading enzymes include cyclomaltodextrinase (or cyclodextrinase, or CDase, EC 3.2.1.54), maltogenic amylase (EC 3.2.1.133), neopullulanase (EC 3.2.1.135), which have been reported to be capable of hydrolyzing CDs and in some cases additional substrates such as pullulan, and starch. Cyclodextrinase (CDase) catalyzes the hydrolysis of CDs to form linear oligosaccharides of α-1,4-linkages, and it can accordingly release substances from CD inclusion complexes. A CDase from Bacillus macerans was reported in 1968, and many CDases from bacteria have since been characterized, such as enzymes from Bacillus sp., Thermoanaerobacter ethanolicus strain 39E, Flavobacterium sp., and Klebsiella oxytoca strain M5a1. Archaea CDases have been characterized from Archaeoglobus fulgidus, Thermococcus sp. B1001, Thermococcus sp. CL1, Thermofilum pendens, and Pyrococcus furiosus. The structure of the CDase from Flavobacterium sp. has been characterized in detail (see Sun et al., Archaea, Volume 2015 (2015), Article ID 397924, reporting the identification of a gene encoding a cyclodextrinase from Thermococcus kodakarensis KOD1 (CDase-Tk)).

Anandamide is a lipidic cannabinoid receptor ligand, the first compound to be identified as an endocannabinoid (also known as (5Z,8Z,11Z,14Z)-N-(2-hydroxyethyl)icosa-5,8,11,14-tetraenamide; N-arachidonoylethanolamine; or, arachidonoylethanolamide). Anandamide analogs in a cyclodextrin inclusion complex have been described as being useful for treating intraocular hypertension (WO1996001558). Anandamide is understood to be a partial agonist of the CB₁ receptor; a weak partial agonist of the CB₂ receptor, a partial agonist of vanilloid receptor VR1 (also known as transient receptor potential cation channel subfamily V member 1; or, TrpV1), and an agonistic ligand of the GPR₅₅ receptor (Reggio P H. Endocannabinoid binding to the cannabinoid receptors: what is known and what remains unknown. Curr Med Chem. 2010; 17 (14): 1468-1486; Roberts L A, Christie M J, Connor M. Anandamide is a partial agonist at native vanilloid receptors in acutely isolated mouse trigeminal sensory neurons. Br J Pharmacol. 2002 October; 137 (4): 421-8). The binding affinity of anandamide and analogs thereof for the CB₁ and CB₂ receptors have been reported, with anandamide evidencing a preferential affinity for CB₁ binding, for example having a Kᵢ for CB₁ of less than 100 nM and a Kᵢ for CB₂ of more than 1000 nM (Lin, S., Khanolkar, A. D., Fan, P., Goutopoulos, A., Qin, C., Papahadjis, D., & Makriyannis, A. (1998). Novel Analogues of Arachidonylethanolamide (Anandamide): Affinities for the CB₁ and CB₂ Cannabinoid Receptors and Metabolic Stability. Journal of Medicinal Chemistry, 41 (27), 5353-5361).

Nitric oxide free radicals are involved in a very wide range of physiological signalling functions, with a complex relationship understood to exist between the endocannabinoid system and nitrergic signalling (Christopher Lipina, Harinder S. Hundal, The endocannabinoid system: ‘NO’ longer anonymous in the control of nitrergic signalling?, Journal of Molecular Cell Biology, Volume 9, Issue 2, April 2017, Pages 91-103). Nitric oxide is synthesized physiologically by a family of nitric oxide synthases that convert L-arginine to L-citrulline and nitric oxide, and L-citrulline is in turn recycled to provide L-arginine. Measurement of fractional NO concentration in exhaled breath (FE_{NO}) has been used as a quantitative, noninvasive, method of measuring airway inflammation. Methods and devices have been described for measuring NO in body fluids, for example by measuring salivary nitric oxide analytes, including nitrite, as a precursor and biomarker for nitric oxide, for example being capable of detecting a concentration range of salivary nitric oxide analyte from 25 to >400 μmol/L nitrite (for example with visibly distinct colorimetric sub-ranges corresponding to: 0 to 25, 25 to 100, 100 to 200, 200 to 350, and greater than 400 μmol/L nitrite (U.S. Pat. No. 9,759,716). Nitric oxide concentrations in body fluids have been suggested to be useful as indicators of physiological anandamide levels (US20190265258). A relatively wide range of perivascular NO concentrations have been reported under control conditions, with values ranging for example from ˜200 to 1,000 nM (Chen K, Pittman R N, Popel A S. Nitric oxide in the vasculature: where does it come from and where does it go? A quantitative perspective. Antioxid Redox Signal. 2008; 10 (7): 1185-1198. doi:10.1089/ars.2007.1959).

Citrulline has been described as being useful in the treatment of a range of conditions associated with NO deficiency (US20010056068). Similarly, L-arginine and other nitric oxide donors have been described as being useful in the treatment of conditions involving NO signalling (EP0441119; U.S. Pat. Nos. 5,595,970 and 5,508,045).

### SUMMARY OF THE INVENTION

Cyclodextrin inclusion complex delivery vehicles are provided in which anandamide or an anandamide analogue are guest molecules. These formulations are provided for use in increasing endogenous nitric oxide levels, and accordingly for treating conditions for which increasing endogenous NO levels has a therapeutic or prophylactic benefit, such as conditions associated with NO deficiency, conditions characterized by anxiety, or an erectile dysfunction. Formulations may for example be used so as to increase measurable NO levels in a subject, or to provide an erectogenic effect in a male subject. With some formulations, these effects may be sustained, for example to achieve sustained NO levels. In addition to anandamide and anandamide analogues, formulations may include additional active agents, optionally in the form of inclusion complex guest molecules, such as NO-generating agents, including citrulline and/or arginine.

A biologically acceptable carrier may be provided for the cyclodextrin inclusion complex, so that the guest molecule is stably retained by the cyclodextrin within the biologically acceptable carrier. An enzyme may also be provided in the vehicle, having a cyclodextrin-degrading activity capable of digesting the cyclodextrin retaining the guest molecule. The enzyme may be formulated so that the cyclodextrin-degrading activity is activated on delivery of the vehicle to a target so as to release the guest molecule from the cyclodextrin cavity.

In alternative aspects of the delivery vehicle, the enzyme may be co-formulated with the cyclodextrin inclusion complex or the enzyme may be co-packaged in the delivery vehicle with the cyclodextrin inclusion complex. When the enzyme is co-packaged, the delivery vehicle may further include a biochemically acceptable carrier for the enzyme.

The enzyme may for example be an amylase, a cyclodextrinase, maltogenic amylase or neopullulanase. An amylase may for example be a mammalian salivary amylase or a pancreatic amylase, or an amylase of fungal, or bacterial origin. A cyclodextrinase may for example be a microbial cyclodextrinase.

The cyclodextrin may for example be a CD derivative, such as a hydrophobic alkylated cyclodextrin or a mixed methylated/ethylated cyclodextrin.

The ratio of the cyclodextrin to the guest molecule may for example be 5:1, 4:1, 3:1, 2:1, 1:1, 1:2, 1:3, 1:4 or 1:5, although a wide range of alternative values for this parameter are also possible, including non-integer ratios.

The cyclodextrin may for example be an alpha, beta or gamma cyclodextrin, although again a very wide range of alternative CD structures may be used. The biologically acceptable carrier may be a pharmaceutically acceptable carrier. The delivery vehicle may be formulated for sustained release of the guest molecules, and/or other active agents. In this way, the invention provides alternative embodiments in which CD delivery vehicles may be formulated and used as a medicaments.

Methods are provided for treating patients having nitric oxide deficiency or a disease which can be treated or prevented by increasing endogenous nitric oxide levels in mammalian subject. Methods are also provided for providing an erectogenic effect in a male subject, for example a male over 50, 60 or 70 years old, or a male subject suffering from an erectile dysfunction.

### DETAILED DESCRIPTION OF THE INVENTION

Anandamide has the following structure:

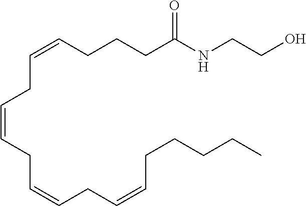 [SMILES: CCCCC/C=C\C/C=C\C/C=C\C/C=C\CCCC(=O)NCCO]

A variety of anandamide analogs are known, including a variety of analogs having a structure of Formula I:

CH₃—(CH₂)ₓ—(CH₂—CH═CH)_{y}—(CH₂)_{z}—C(═O′)—N′(R¹)(R²)

wherein:
x is an integer from 1 to 6;
y is an integer from 1 to 6;
z in an integer from 1 to 6;
R¹ and R² are independently selected from the group consisting of: H; C₁₋₆ alkyl; and (CH₂)_{w}—R³, wherein w is an integer from 0 to 6; and,
R³ is selected from the group consisting of: CH₃, OH, SH, F, Cl, Br, I, C≡CH, C≡N, a carbocyclic ring having from 3 to 7 carbons, and a heterocyclic ring having from 3 to 7 carbons and at least one heteroatom selected from N, O and S; and,
wherein R¹ and/or R² may be combined with N′ or O′ to form a heterocyclic ring having 3 to 7 atoms.

Anandamide analogs for use in the present formulations may be characterized by receptor binding activities, for example being one or more of: partial agonists of the CB₁ receptor; weak partial agonists of the CB₂ receptor, partial agonists of vanilloid receptor VR1; and/or an agonistic ligand of the GPR₅₅ receptor. Alternatively, the binding affinity of anandamide analogs for the CB₁ and CB₂ receptors may be characterized by a preferential affinity for CB₁ binding, for example having a Kᵢ for CB₁ of less than 100 nM and a Kᵢ for CB₂ of more than 1000 nM.

The present formulations may be used so as to raise physiological NO levels, for example for treating or preventing a nitric oxide deficiency or a disease which can be treated or prevented by increasing endogenous nitric oxide levels in a mammal. It is understood that deficiencies in nitric oxide contribute to the pathogenesis of cardiovascular system conditions, including hypertension and the cardiovascular disease (e.g. atherosclerosis, restenosis). It is understood that in female human patients, NO deficiency may be involved in pathological conditions such as preeclampsia, preterm birth, cervical incompetence, recurrent abortions, dysmenorrhea, infertility, hot flushes, cardiovascular disease, urinary incontinence and cognition problems. Similarly, particularly in aging men, NO deficiency is implicated in cardiovascular disease, hypertension, impotence and osteoporosis. Accordingly, treatments are provided herein for nitric oxide-associated disorders and diseases such as hypertension, cardiovascular disease (e.g. atherosclerosis, restenosis), osteoporosis, preeclampsia, preterm labor, dysmenorrhea, cervical dystocia, urinary incontinence, male impotence, and female infertility.

Select embodiments of the present formulations include citrulline or a citrulline analogue, such as: D,L-citrulline, L-citrulline, L-citrulline monoacetate, L-citrulline hydrochloride, L-citrulline methylester, L-citrulline ethylester, L-citrulline-n-hexylester, L-citrulline (benzoylmethyl) ester, alpha-N-benzoyl-L-citrulline methylester, N-Boc-L-citrulline, or N1-2,4-dinitrophenyl-D,L-citrulline. In alternative embodiments, citrulline analogues may have the structure of Formula II:

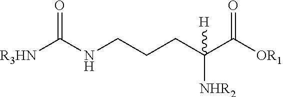

wherein:
R¹ is hydrogen, C₁₋₁₀ alkyl, C₁₋₁₀ alkenyl, aryl, —CH₂)₁₋₃(C═O) aryl, omega-hydroxyalkyl or omega-methoxyalkyl,
R² and R³ are selected independently from hydrogen, C₁₋₁₀ alkyl, aryl, acetyl, benzoyl, and tert-butoxycarbonyl.

Specific formulations may for example include cyclodextrin inclusion complex formulations made by combining cyclodextrin inclusions of anandamide or an analogue thereof co-formulated with a CD degrading enzyme.

In select embodiments, the enzyme provided in the vehicle may be formulated so that the cyclodextrin-degrading activity is activated on delivery of the vehicle to a target so as to release the guest molecule from the cyclodextrin cavity. Enzyme activation may for example be accomplished in a medicament, for example for oral delivery, in a dry dosage form, such as a capsule or tablet, in which the enzyme is admixed, so that the enzyme will not be active until activated by moisture in the gastrointestinal tract of a host. Similarly, a wide variety of time release matrices and formulations are known, which may be adapted for use in CD delivery vehicles so as to orchestrate the appropriate activation of the CD-degrading enzyme upon delivery to the target.

In various aspects, CD delivery vehicles may have the enzyme co-formulated with the cyclodextrin inclusion complex, as for example discussed above, or the enzyme may be co-packaged in the delivery vehicle with the cyclodextrin inclusion complex. In the case of co-packaging, the delivery vehicle may for example include a biochemically acceptable carrier for the enzyme-distinct from the carrier for the CD inclusion complex. For example, delivery vehicles may be provided with separated compartments containing the CD inclusion complex and the CD-degrading enzyme, so that the delivery vehicle will be made up of a CD inclusion complex compartment connected to a CD-degrading enzyme compartment. Mechanisms may be provided for the combined release of the CD inclusion complex and the CD-degrading enzyme from the respective compartments in the delivery vehicle. For example, syringes may be provided having distinct compartments of this kind that are discharged by a common discharge mechanism, such as a mechanism that cooperatively displaces pistons in each compartment so as to discharge aliquots of CD inclusion complex and CD-degrading enzyme, so that the enzyme and the complex may then be comingled to activate the enzymatic release of the guest molecule from the CD. Vehicles of this kind may for example be used to dispense a topical cream or other surface-active formulations. A wide variety of delivery vehicles of this kind may be adapted from devices that are known for dispensing two-part compositions such as epoxy resins, two-part medicaments or dental formulations, as for example disclosed in U.S. Pat. Nos. 4,538,920, 8,100,295, 8,308,340, 8,875,947, 8,499,976 and International Patent Publications WO2007041266 and WO2000021842.

There are a wide variety of techniques available to prepare CD inclusion complexes, as for example described in: Chaudhary & Patel, IJPSR, 2013, Vol. 4 (1): 68-76; Carneiro et al., 2019, Int. J. Mol. Sci. 2019, 20, 642; US Patent Publications US20090029020; US20090214446; U.S. Pat. Nos. 5,070,081; 5,552,378; 5,674,854, and 8,658,692. A common approach is known as the kneading method, which involves mixing CDs with water or an aqueous alcohol to provide a paste. The bioactive molecule may then be added to the paste and kneaded for a specified time. The kneaded mixture may then be dried and passed through sieve if desired. Alternatively, a slurry method involves steps of: mixing the bioactive molecule and the cyclodextrin, adding a suitable amount of water to the mixture, typically with vigorous mixing, until a paste or a slurry is formed; continuing the mixing with further addition of water if necessary to maintain the paste or the slurry consistency, for a suitable period of time, such as 15 minutes, to form the inclusion complex; and, drying the product of this final step. Other ingredients, such as emulsifiers, may facilitate the formation of inclusion complexes, for example processes that involve steps of: dry blending a cyclodextrin and an emulsifier (e.g., pectin); combining the dry blend of cyclodextrin and the emulsifier with a solvent such as water in a reactor, and agitating; adding the guest molecule and stirring (e.g., for approximately 5 to 8 hours); optionally cooling the reaction mixture with stirring; and emulsifying the mixture prior to drying the cyclodextrin inclusion complex to form a powder. Other known approaches to preparing CD inclusions involve lyophilization, microwave irradiation, and a supercritical fluid antisolvent technique.

The CD delivery vehicles of the invention can be provided alone or in combination with other compounds (for example, nucleic acid molecules, small molecules, peptides, or peptide analogues), in the presence of a carrier, such as a liposome, an adjuvant, or any pharmaceutically or biologically acceptable carrier. Select embodiments include medicaments in a form suitable for administration to animal hosts, such as mammals, for example, humans. As used herein “pharmaceutically acceptable carrier” or “excipient” includes any and all solvents, dispersion media, coatings, antibacterial and antifungal agents, isotonic and absorption delaying agents, and the like that are physiologically compatible. The carrier can be suitable for any appropriate form of administration, including topical, subcutaneous, intradermal, intravenous, parenteral, intraperitoneal, intramuscular, sublingual, inhalational, intratumoral or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the biologically active compound, use thereof in the pharmaceutical compositions of the invention is contemplated. Supplementary active compounds can also be incorporated into the compositions.

Conventional pharmaceutical practice may be employed to provide suitable formulations or compositions to administer the delivery vehicles to subjects. Any appropriate route of administration may be employed, for example, parenteral, intravenous, intradermal, subcutaneous, intramuscular, intracranial, intraorbital, ophthalmic, intraventricular, intracapsular, intraspinal, intrathecal, intracisternal, intraperitoneal, intranasal, inhalational, aerosol, topical, intratumoral, sublingual or oral administration. Therapeutic formulations may be in the form of liquid solutions or suspensions; for oral administration, formulations may be in the form of tablets or capsules; for intranasal formulations, in the form of powders, nasal drops, or aerosols; and for sublingual formulations, in the form of drops, aerosols or tablets.

Cyclodextrin-degrading or digesting enzymes may for example be formulated for oral delivery. Enteric enzyme formulations may for example be provided, such as submicron particle formulations prepared by emulsion solvent evaporation (Sharma et al., Pharm Dev Technol. 2013 May-June; 18 (3): 560-9). Similarly, delivery vehicles may be formulated as hydrogels (see US Patent Publication 20140094433), or medicated gums (see US Patent Publication 20130022652).

Methods well known in the art for making formulations are found in, for example, “Remington's Pharmaceutical Sciences” (20th edition), ed. A. Gennaro, 2000, Mack Publishing Company, Easton, PA. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Pharmaceutical compositions of the present invention may be in any form which allows for the composition to be administered to a patient. For example, the composition may be in the form of a solid, liquid or gas (aerosol). Typical routes of administration include, without limitation, oral, topical, parenteral, sublingual, rectal, vaginal, and intranasal. The term parenteral as used herein includes subcutaneous injections, intravenous, intramuscular, epidural, intrasternal injection or infusion techniques. Pharmaceutical composition of the invention are formulated so as to allow the active ingredients contained therein to be bioavailable upon administration of the composition to a patient. Compositions that will be administered to a patient take the form of one or more dosage units, where for example, a tablet, capsule or cachet may be a single dosage unit, and a container of the compound in aerosol form may hold a plurality of dosage units.

Materials used in preparing the pharmaceutical compositions should be pharmaceutically pure and non-toxic in the amounts used. The inventive compositions may include one or more compounds (active ingredients) known for a particularly desirable effect. It will be evident to those of ordinary skill in the art that the optimal dosage of the active ingredient(s) in the pharmaceutical composition will depend on a variety of factors. Relevant factors include, without limitation, the type of subject (e.g., human), the particular form of the active ingredient, the manner of administration and the composition employed.

In general, the pharmaceutical composition includes a delivery vehicle of the present invention as described herein, in admixture with one or more carriers. The carrier(s) may be particulate, so that the compositions are, for example, in tablet or powder form. The carrier(s) may be liquid, with the compositions being, for example, an oral syrup or injectable liquid. In addition, the carrier(s) may be gaseous, so as to provide an aerosol composition useful in, e.g., inhalatory administration.

When intended for oral administration, the composition is preferably in either solid or liquid form, where semi-solid, semi-liquid, suspension and gel forms are included within the forms considered herein as either solid or liquid.

As a solid composition for oral administration, the composition may be formulated into a powder, granule, compressed tablet, pill, capsule, cachet, chewing gum, wafer, lozenges, or the like form. Such a solid composition will typically contain one or more inert diluents or edible carriers. In addition, one or more of the following adjuvants may be present: binders such as syrups, acacia, sorbitol, polyvinylpyrrolidone, carboxymethylcellulose, ethyl cellulose, microcrystalline cellulose, gum tragacanth or gelatin, and mixtures thereof; excipients such as starch, lactose or dextrins, disintegrating agents such as alginic acid, sodium alginate, Primogel, corn starch and the like; lubricants such as magnesium stearate or Sterotex; fillers such as lactose, mannitols, starch, calcium phosphate, sorbitol, methylcellulose, and mixtures thereof; lubricants such as magnesium stearate, high molecular weight polymers such as polyethylene glycol, high molecular weight fatty acids such as stearic acid, silica, wetting agents such as sodium lauryl sulfate, glidants such as colloidal silicon dioxide; sweetening agents such as sucrose or saccharin, a flavoring agent such as peppermint, methyl salicylate or orange flavoring, and a coloring agent.

When the composition is in the form of a capsule, e.g., a gelatin capsule, it may contain, in addition to materials of the above type, a liquid carrier such as polyethylene glycol or a fatty oil.

The composition may be in the form of a liquid, e.g., an elixir, syrup, solution, aqueous or oily emulsion or suspension, or even dry powders which may be reconstituted with water and/or other liquid media prior to use. The liquid may be for oral administration or for delivery by injection, as two examples. When intended for oral administration, preferred compositions contain, in addition to the present compounds, one or more of a sweetening agent, thickening agent, preservative (e.g., alkyl p-hydoxybenzoate), dye/colorant and flavor enhancer (flavorant). In a composition intended to be administered by injection, one or more of a surfactant, preservative (e.g., alkyl p-hydroxybenzoate), wetting agent, dispersing agent, suspending agent (e.g., sorbitol, glucose, or other sugar syrups), buffer, stabilizer and isotonic agent may be included. The emulsifying agent may be selected from lecithin or sorbitol monooleate.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

The pharmaceutical composition may be intended for topical administration, in which case the carrier may suitably comprise a solution, emulsion, ointment, cream or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the biologically active compound of from about 0.1 to about 25% w/v (weight per unit volume).

The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol. Low-melting waxes are preferred for the preparation of a suppository, where mixtures of fatty acid glycerides and/or cocoa butter are suitable waxes. The waxes may be melted, and the aminocyclohexyl ether compound is dispersed homogeneously therein by stirring. The molten homogeneous mixture is then poured into convenient sized molds, allowed to cool and thereby solidify.

The composition may include various materials which modify the physical form of a solid or liquid dosage unit. For example, the composition may include materials that form a coating shell around the active ingredients. The materials which form the coating shell are typically inert, and may be selected from, for example, sugar, shellac, and other enteric coating agents. Alternatively, the active ingredients may be encased in a gelatin capsule or cachet.

The pharmaceutical composition of the present invention may consist of gaseous dosage units, e.g., it may be in the form of an aerosol. The term aerosol is used to denote a variety of systems ranging from those of colloidal nature to systems consisting of pressurized packages. Delivery may be by a liquefied or compressed gas or by a suitable pump system which dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit.

The biologically active compounds may be in the form of the free base or in the form of a pharmaceutically acceptable salt such as the hydrochloride, sulfate, phosphate, citrate, fumarate, methanesulfonate, acetate, tartrate, maleate, lactate, mandelate, salicylate, succinate and other salts known in the art. The appropriate salt would be chosen to enhance bioavailability or stability of the compound for the appropriate mode of employment (e.g., oral or parenteral routes of administration).

A composition intended to be administered by injection can be prepared by combining the delivery vehicle of the present invention with water, and preferably buffering agents, so as to form a solution. The water is preferably sterile pyrogen-free water. A surfactant may be added to facilitate the formation of a homogeneous solution or suspension. Surfactants are compounds that non-covalently interact with the aminocyclohexyl ether compound so as to facilitate dissolution or homogeneous suspension of the aminocyclohexyl ether compound in the aqueous delivery system. Surfactants are desirably present in aqueous compositions of the invention because the aminocyclohexyl ether compounds according to the present invention may be hydrophobic. Other carriers for injection include, without limitation, sterile peroxide-free ethyl oleate, dehydrated alcohols, propylene glycol, as well as mixtures thereof.

Suitable pharmaceutical adjuvants for the injecting solutions include stabilizing agents, solubilizing agents, buffers, and viscosity regulators. Examples of these adjuvants include ethanol, ethylenediaminetetraacetic acid (EDTA), tartrate buffers, citrate buffers, and high molecular weight polyethylene oxide viscosity regulators. These pharmaceutical formulations may be injected intramuscularly, epidurally, intraperitoneally, or intravenously.

The present invention also provides kits that contain a pharmaceutical composition which includes one or more delivery vehicles. The kit also includes instructions for the use of the pharmaceutical. Preferably, a commercial package will contain one or more unit doses of the pharmaceutical composition. For example, such a unit dose may be an amount sufficient for the preparation of an intravenous injection. It will be evident to those of ordinary skill in the art that compounds which are light and/or air sensitive may require special packaging and/or formulation. For example, packaging may be used which is opaque to light, and/or sealed from contact with ambient air, and/or formulated with suitable coatings or excipients.

An “effective amount” of a CD inclusion complex delivery vehicle according to the invention includes a therapeutically effective amount or a prophylactically effective amount. A “therapeutically effective amount” refers to an amount effective, at dosages and for periods of time necessary, to achieve the desired therapeutic result. A therapeutically effective amount of a delivery vehicle may vary according to factors such as the disease state, age, sex, and weight of the individual, and the ability of the compound to elicit a desired response in the individual. Dosage regimens may be adjusted to provide the optimum therapeutic response. A therapeutically effective amount may also be one in which any toxic or detrimental effects of the delivery vehicle or active compound are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. For any particular subject, the timing and dose of treatments may be adjusted over time (e.g., timing may be daily, every other day, weekly, monthly) according to the individual need and the professional judgment of the person administering or supervising the administration of the compositions.

In select embodiments, the present invention provides a composition or medicament that includes one or more biologically active molecules, selected from biologically active compounds or a solvate, pharmaceutically acceptable salt, ester, amide, complex, chelate, stereoisomer, stereoisomeric mixture, geometric isomer, crystalline or amorphous form, metabolite, metabolic precursor or prodrug thereof, including isolated enantiomeric, diastereomeric and geometric isomers thereof, and mixtures thereof, in combination with a pharmaceutically acceptable carrier, diluent or excipient, and further provides a method for the manufacture of such a composition or medicament.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing.

Citation of references herein is not an admission that such references are prior art to the present invention. Any priority document(s) and all publications, including but not limited to patents and patent applications, cited in this specification are incorporated herein by reference. All documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual publication were specifically and individually indicated to be incorporated by reference herein and as though fully set forth herein. The invention includes all embodiments and variations substantially as hereinbefore described and with reference to the examples and drawings.

In some embodiments, the invention excludes steps that involve medical or surgical treatment.

### EXAMPLES

Exemplary formulations that have been tested in subjects are set out below.

Anandamide, mw 347, was prepared for formulating by inclusion in gamma cyclodextrin, at a 1:2 ratio, with anandamide provided at 11.5 wt % of the formulation as an 80% anandamide oil, providing a final anandamide concentration of ˜9.2 wt %. Alternative anandamide CD inclusion formulations were prepared similarly at 7.7 wt % anandamide and 8.8 wt % anandamide.

Capsules were prepared from the anandamide inclusion formulation as follows:

### 42 mg Capsules

465 mg anandamide 9.2% CD inclusion, 42 mg net anandamide
30 mg E4M cellulose (hydroxypropylmethylcellulose, to deliver 1-2 hour release)
4 mg amylase
5 mg Ca laurate (flow agent)

### 10 mg Capsules

130 mg anandamide 7.7% CD inclusion
40 mg E4M cellulose
335 mg serine
5 mg amylase
2 mg sodium stearyl fumarate (SSF-flow agent)

### 5 mg Capsules

65 anandamide 7.7% CD inclusion
400 mg serine
5 mg amylase
2 mg SSF

### 25 mg Capsules

325 mg anandamide 7.7% CD inclusion with amylase
30 mg E4M cellulose
230 mg serine
2 mg SSF

### Example 1

A male subject was treated with one 42 mg capsule in morning, and one 10 mg capsule in the evening. The subject reported that this treatment completely neutralized stress, and helped with sleep. After some time, the subject continued with a dose of two 10 mg capsules in the morning, and no treatments in the evening, with the result of continued stress relief.

Subsequently, the subject began taking two 25 mg capsules in the morning, with the result a surprising increase in blood flow when tumescent. The subject had been taking various amounts of sildenafil for many years at 10-25 mg increments. In conjunction with the 25 mg dosing of the anandamide CD formulation, the subject noted that 10 mg of sildenafil would produce more noticeable results than a 25 mg dose taken before anandamide CD supplementation. The subject noted that the anandamide CD formulation had a noticeable cumulative effect over time, with blood flow improving with prolonged treatment.

The subject subsequently changed dosing to one 25 mg capsule in the morning and one 10 mg capsule in the afternoon. In past salivary NO testing using commercially available nitrite test strips (as described in U.S. Pat. No. 9,759,716) the subject had never had an indication above “depleted” or “low” on a NO color indicator strip. However, at this stage of anandamide CD treatment, the NO test strips now provided an indication well above “low” on the color scale, and reaching “optimal” or better in color intensity.

### Example 2

This example relates to the use of two alternative formulations, augmented with the nitric oxide synthase substrate arginine, and with citrulline which supports the sustained formation of arginine:

### 10 mg Sustained Release Anandamide (SR)

120 mg anandamide 8.5% CD inclusion with amylase in base
72 mg K250 cellulose (sustained release 10-12 hours)
120 mg citrulline (NO amino acid)
100 mg arginine (NO amino acid)
5 mg Ca laurate

### 10 Mg Quick Release Anandamide (QR)

120 mg anandamide 8.5% CD inclusion with amylase in base
15 mg E4M cellulose (sustained release 10-12 hours)
160 mg citrulline (NO amino acid)
120 mg arginine (NO amino acid)
5 mg Ca laurate

The subject of Example 1 determined that the combined use of these formulations permitted controlled dosing with bolus and/extended release regimens. This combination therapy provided treatment results that were even more compelling for NO production and consistency as well as erectile performance. Taking 2 of the SR capsules and one QR capsule in the morning and one QR capsule in the afternoon and 2 SR capsules at about 6 p.m. left the subject with an almost painful erection, after stimulation, at about 9:30 p.m. that night.

### Example 3

Subject EL self administered a daily 500 mg dosage form for approximately 4 months, of the following formula:

### 500 mg Capsules

160 mg anandamide 7.8% CD inclusion, 12.5 mg net anandamide
300 mg citrulline
20 mg E4M cellulose (hydroxypropylmethylcellulose, to deliver 1-2 hour release)
5 mg amylase
15 mg Ca laurate (flow agent)

Subject EL reported a noticeable decrease in anxiety and stress, as well as far better sleep, a 2 point increase in VO₂ Max when running (as measured on a Garmin device), a noticeable increase in lung capacity, drop in heart rate at high intensity and improvement in running performance, estimated at a 10-15% improvement. This example illustrates the use of the cyclodextrin (CD) inclusion complex formulation to increase cardiovascular performance of a human subject.

1. A method of treating or preventing a nitric oxide deficiency or a disease which can be treated or prevented by increasing endogenous nitric oxide levels in a mammal, or of increasing endogenous nitric oxide levels in a mammal, or of increasing the cardiovascular performance of a mammal, comprising orally administering to the mammal an effective amount of a cyclodextrin (CD) inclusion complex formulation, comprising: anandamide or an analogue thereof as a guest molecule in the CD; and, a co-formulated cyclodextrin degrading enzyme.

2. The method of claim 1, further comprising administering an effective amount of a nitric oxide synthase substrate.

3. The method of claim 2, wherein the nitric oxide synthase substrate is L-arginine.

4. The method of any one of claims 1 to 3, further comprising administering an effective amount of citrulline or a citrulline analogue.

5. The method of claim 4, wherein the citrulline analogue is D,L-citrulline, L-citrulline, L-citrulline monoacetate, L-citrulline hydrochloride, L-citrulline methylester, L-citrulline ethylester, L-citrulline-n-hexylester, L-citrulline (benzoylmethyl) ester, alpha-N-benzoyl-L-citrulline methylester, N-Boc-L-citrulline, or N1-2,4-dinitrophenyl-D,L-citrulline.

6. The method of claim 4, wherein the citrulline analogue has the structure of Formula II: 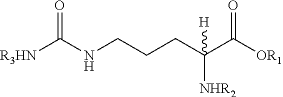
wherein:
R¹ is hydrogen, C₁₋₁₀ alkyl, C₁₋₁₀ alkenyl, aryl, —CH₂)₁₋₃(C═O) aryl, omega-hydroxyalkyl or omega-methoxyalkyl,
R² and R³ are selected independently from hydrogen, C₁₋₁₀ alkyl, aryl, acetyl, benzoyl, and tert-butoxycarbonyl.

7. The method of any one of claims 1 to 6, wherein the anandamide analogue has a structure of Formula I:
CH₃—(CH₂)ₓ—(CH₂—CH═CH)_{y}—(CH₂)_{z}—C(═O′)—N′(R¹)(R²)
wherein:
x is an integer from 1 to 6;
y is an integer from 1 to 6;
z in an integer from 1 to 6;
R¹ and R² are independently selected from the group consisting of: H; C₁₋₆ alkyl; and
(CH₂)_{w}—R³, wherein w is an integer from 0 to 6; and,
R³ is selected from the group consisting of: CH₃, OH, SH, F, Cl, Br, I, C≡CH, C≡N, a carbocyclic ring having from 3 to 7 carbons, and a heterocyclic ring having from 3 to 7 carbons and at least one heteroatom selected from N, O and S; and,
wherein R¹ and/or R² may be combined with N′ or O′ to form a heterocyclic ring having 3 to 7 atoms.

8. The method of claim 7, wherein the anandamide analogue is: a partial agonist of the CB₁ receptor; a weak partial agonist of the CB₂ receptor, a partial agonist of vanilloid receptor VR1, and/or an agonistic ligand of the GPR₅₅ receptor.

9. The method of claim 7 or 8, wherein the anandamide analogue has a preferential affinity for CB₁ binding over CB₂ binding.

10. The method of claim 9, wherein the anandamide analogue has a Kᵢ for CB₁ of less than 100 nM and a Kᵢ for CB₂ of more than 1000 nM.

11. The method of any one of claims 1-10, wherein the mammal is a human, and wherein the disease is atherosclerosis, restenosis, hypertension, preeclampsia, female infertility, cervical dystocia, pyloric stenosis, diabetes mellitus, asthma, neonatal respiratory distress syndrome, adult respiratory distress syndrome, acute inflammation, SLE-lupus, anaphylactic reaction, allograft rejection, Alzheimer's disease, stroke, anxiety or erectile disfunction.

12. The method of any one of claims 1-11, wherein the NO deficiency is characterized by a measured level of salivary nitrite of less than 100, 200, 250, 300, or 350 umol/L nitrite, or a salivary nitrite level of 0 to 25 or 25 to 100 umol/L nitrite.

13. The method of any one of claims 1-12, wherein the CD inclusion complex formulation of further comprises a time release agent.

14. The method of claim 13, wherein the time release agent is hydroxypropylmethylcellulose.

15. The method of any one of claims 1-14, wherein the cyclodextrin degrading enzyme is an amylase, a cyclodextrinase, a microbial cyclodextrinase, a maltogenic amylase or neopullulanase.

16. The method according to claim 15, wherein the amylase is a mammalian salivary amylase, a mammalian pancreatic amylase or a microbial amylase.

17. The method of any one of claims 1-16, further comprising a pharmaceutically acceptable carrier.

18. The method of claim 17, wherein the pharmaceutically acceptable carrier is calcium laurate.

19. The method of any one of claims 1-18, wherein the enzyme is formulated so that a cyclodextrin-degrading activity is activated on delivery of the formulation to a target tissue so as to release guest molecules from the cyclodextrin, optionally wherein the CD inclusion complex formulation is administered orally, and the target tissue is a mammalian gastrointestinal (GI) tract, optionally a human GI tract.

20. The method according to any one of claims 1-19, wherein the cyclodextrin comprises an alpha cyclodextrin, beta cyclodextrin or gamma cyclodextrin.

21. The method according to any one of claims 1-20, wherein the cyclodextrin is a gamma cyclodextrin.

22. The method according to any one of claims 1-21, wherein the cyclodextrin is a mixed methylated/ethylated cyclodextrin or a hydrophobic alkylated cyclodextrin.

23. The method according to any one of claims 1-22, wherein the formulation further comprises one or more additional CD guest molecules.

24. The method according to any one of claims 1-23, wherein the ratio of the cyclodextrin to the anandamide or anandamide analogue is from 5:1 to 1:5.

25. The method according to any one of claims 1-24, wherein the formulation is formulated for sustained release of the anandamide or analogue thereof.

26. The method according to any one of claims 1-25, wherein the disease is erectile dysfunction.

27. A cyclodextrin (CD) inclusion complex formulation, comprising: anandamide or an analogue thereof as a guest molecule in the CD; and, a co-formulated cyclodextrin degrading enzyme.

28. The CD inclusion complex formulation of claim 27, further comprising an effective amount of a nitric oxide synthase substrate.

29. The CD inclusion complex formulation of claim 28, wherein the nitric oxide synthase substrate is L-arginine.

30. The CD inclusion complex formulation of any one of claims 27 to 29, further comprising an effective amount of citrulline or a citrulline analogue.

31. The CD inclusion complex formulation of claim 30, wherein the citrulline analogue is D,L-citrulline, L-citrulline, L-citrulline monoacetate, L-citrulline hydrochloride, L-citrulline methylester, L-citrulline ethylester, L-citrulline-n-hexylester, L-citrulline (benzoylmethyl) ester, alpha-N-benzoyl-L-citrulline methylester, N-Boc-L-citrulline, or N1-2,4-dinitrophenyl-D,L-citrulline.

32. The CD inclusion complex formulation of claim 30, wherein the citrulline analogue has the structure of Formula II: 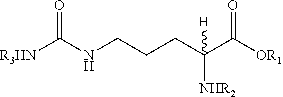
wherein:
R¹ is hydrogen, C₁₋₁₀ alkyl, C₁₋₁₀ alkenyl, aryl, —CH₂)₁₋₃(C═O) aryl, omega-hydroxyalkyl or omega-methoxyalkyl,
R² and R₅ are selected independently from hydrogen, C₁₋₁₀ alkyl, aryl, acetyl, benzoyl, and tert-butoxycarbonyl.

33. The CD inclusion complex formulation of any one of claims 27 to 32, wherein the anandamide analogue has a structure of Formula I:
CH₃—(CH₂)ₓ—(CH₂—CH═CH)_{y}—(CH₂)_{z}—C(═O′)—N′(R¹)(R²)
wherein:
x is an integer from 1 to 6;
y is an integer from 1 to 6;
z in an integer from 1 to 6;
R¹ and R² are independently selected from the group consisting of: H; C₁₋₆ alkyl; and
(CH₂)_{w}—R³, wherein w is an integer from 0 to 6; and,
R³ is selected from the group consisting of: CH₃, OH, SH, F, Cl, Br, I, C≡CH, C≡N, a carbocyclic ring having from 3 to 7 carbons, and a heterocyclic ring having from 3 to 7 carbons and at least one heteroatom selected from N, O and S; and,
wherein R¹ and/or R² may be combined with N′ or O′ to form a heterocyclic ring having 3 to 7 atoms.

34. The CD inclusion complex formulation of claim 33, wherein the anandamide analogue is: a partial agonist of the CB₁ receptor; a weak partial agonist of the CB₂ receptor, a partial agonist of vanilloid receptor VR1, and/or an agonistic ligand of the GPR₅₅ receptor.

35. The CD inclusion complex formulation of claim 32 or 33, wherein the anandamide analogue has a preferential affinity for CB₁ binding over CB₂ binding.

36. The CD inclusion complex formulation of claim 35, wherein the anandamide analogue has a Kᵢ for CB₁ of less than 100 nM and a Kᵢ for CB₂ of more than 1000 nM.

37. The CD inclusion complex formulation of any one of claims 27-36, for use in increasing endogenous nitric oxide levels in a mammal, increasing cardiovascular performance of the mammal, or for treating or preventing a nitric oxide deficiency or a disease which can be treated or prevented by increasing endogenous nitric oxide levels in the mammal, or for treating a human disease that is atherosclerosis, restenosis, hypertension, preeclampsia, female infertility, cervical dystocia, pyloric stenosis, diabetes mellitus, asthma, neonatal respiratory distress syndrome, adult respiratory distress syndrome, acute inflammation, SLE-lupus, anaphylactic reaction, allograft rejection, Alzheimer's disease, stroke, anxiety or erectile disfunction.

38. The CD inclusion complex formulation of any one of claims 27-37, for use in treating a NO deficiency that is characterized by a measured level of salivary nitrite of less than 100, 200, 250, 300, or 350 umol/L nitrite, or a salivary nitrite level of 0 to 25 or 25 to 100 umol/L nitrite.

39. The CD inclusion complex formulation of any one of claims 27-38, wherein the CD inclusion complex formulation of further comprises a time release agent.

40. The CD inclusion complex formulation of claim 39, wherein the time release agent is hydroxypropylmethylcellulose.

41. The CD inclusion complex formulation of any one of claims 27-40, wherein the cyclodextrin degrading enzyme is an amylase, a cyclodextrinase, a microbial cyclodextrinase, a maltogenic amylase or neopullulanase.

42. The CD inclusion complex formulation according to claim 41, wherein the amylase is a mammalian salivary amylase, a mammalian pancreatic amylase or a microbial amylase.

43. The CD inclusion complex formulation of any one of claims 27-42, further comprising a pharmaceutically acceptable carrier.

44. The CD inclusion complex formulation of claim 43, wherein the pharmaceutically acceptable carrier is calcium laurate.

45. The CD inclusion complex formulation of any one of claims 27-44, wherein the enzyme is formulated so that a cyclodextrin-degrading activity is activated on delivery of the formulation to a target tissue so as to release guest molecules from the cyclodextrin, optionally wherein the CD inclusion complex formulation is formulated for oral use, and the target tissue is a mammalian gastrointestinal (GI) tract, optionally a human GI tract.

46. The CD inclusion complex formulation according to any one of claims 27-45, wherein the cyclodextrin comprises an alpha cyclodextrin, beta cyclodextrin or gamma cyclodextrin.

47. The CD inclusion complex formulation according to any one of claims 27-46, wherein the cyclodextrin is a gamma cyclodextrin.

48. The CD inclusion complex formulation according to any one of claims 27-47, wherein the cyclodextrin is a mixed methylated/ethylated cyclodextrin or a hydrophobic alkylated cyclodextrin.

49. The CD inclusion complex formulation according to any one of claims 27-48, wherein the formulation further comprises one or more additional CD guest molecules.

50. The CD inclusion complex formulation according to any one of claims 27-49, wherein the ratio of the cyclodextrin to the anandamide or anandamide analogue is from 5:1 to 1:5.

51. The CD inclusion complex formulation according to any one of claims 27-50, wherein the formulation is formulated for sustained release of the anandamide or analogue thereof.

52. The method of any one of claims 1-26 or the CD inclusion complex formulation of any one of claims 27-51, wherein the mammal is a human.
